(19) United States
(12) Patent Application Publication
(10) Pub. No.: US 2026/0198509 A1
(43) Pub. Date: Jul. 16, 2026

(54) DEVICE AND METHOD FOR PREDICTING STORAGE PERIOD OF FRUITS

(71) Applicant: Chinese Academy of Agricultural Sciences Fruit Research Institute, Huludao (CN)

(72) Inventors: Zhihua Wang, Huludao (CN); Wenhui Wang, Huludao (CN); Chaoshuang Jia, Huludao (CN); Wenkuan Zhang, Huludao (CN)

(21) Appl. No.: 19/318,805

(22) Filed: Sep. 4, 2025

(30) Foreign Application Priority Data
Jan. 14, 2025 (CN) 202510056122.0

(51) Int. Cl. A23B 7/148; A23B 2/00; A23B 2/717; B65D 81/26; B65D 85/34; G01N 33/02; H04N 23/50

(52) U.S. Cl. CPC A23B 7/148; A23B 2/001; A23B 2/717; B65D 81/266; B65D 85/34; G01N 33/025; H04N 23/555

(57) ABSTRACT

A device for predicting fruit storage periods includes an adjustment prediction assembly arranged on an outer side of each storage box, and a switching auxiliary assembly mounted inside each storage box and the bottom support box. A method for predicting fruit storage periods includes the steps of: S1, inspection preparation; S2, placement and stacking; S3, inspection and prediction; and S4, environmental modification. Through optical observation, fruit changes are monitored to assess current fruit conditions, and an initially predicted storage period is adjusted. With this simple operation, air is dried by silica gel desiccant and deoxygenated by iron powder in an annular drying cage, and the original air within the storage boxes is exhausted, creating a low-oxygen, dry environment and extending the predicted fruit storage period.

### CROSS-REFERENCE TO RELATED APPLICATION

This application claims priority of Chinese Patent Application No. 202510056122.0, filed on Jan. 14, 2025, the entire contents of which are incorporated herein by reference.

### TECHNICAL FIELD

The present disclosure relates to the technical field of fruit storage prediction, and specifically to a device and method for predicting the storage period of fruits.

### BACKGROUND

With rising urban living standards, consumers demand increasingly stringent levels of safety and freshness in agricultural products. However, given the inherent characteristics of fruits and the geographic dispersion between production and consumption regions, harvested fruits require simple processing followed by storage and distribution to other markets and processing facilities. Before storage, a storage period prediction is essential to mitigate prolonged transit risks that may cause spoilage before reaching destination points. Currently, a real-time inspection of fruit conditions is performed for prediction.

Chinese patent application No. 202420623356. X provides “A fruit inspection device”. This device cooperates with motorized guide rails to achieve multi-directional dimensional inspection of fruits, thereby reducing human-induced measurement deviations that cause statistical inaccuracies. It solves problems in conventional equipment, which can only detect single parameters of fruits, resulting in incomplete inspection results. However, for the above device, after inspection and prediction of the storage period, the accuracy of the predicted storage period is compromised by variable transit environments and fluctuating storage conditions, necessitating labor-intensive spot inspections during transportation, thereby increasing operational complexity and time consumption.

### SUMMARY

The present disclosure provides a device and method for predicting the storage period of fruits, which can effectively solve the problem mentioned in the background that after inspection and prediction of the storage period, the accuracy of the predicted storage period is compromised by variable transit environments and fluctuating storage conditions, necessitating labor-intensive spot inspections during transportation, thereby increasing operational complexity and time consumption.

To realize the above objective, the present disclosure provides the following technical solutions: a device for predicting the storage period of fruits includes storage boxes, an adjustment prediction assembly is arranged on an outer side of each storage box, and the adjustment prediction assembly includes a bottom support ring;

a bottom end of the storage box is bonded to the bottom support ring, a bottom support box is movably sleeved over an outer side of the bottom support ring, a ventilation hole is disposed on one side of the bottom support box, a ventilation pipe is embedded and mounted inside the ventilation hole, the ventilation pipe penetrates through a middle part of a bottom support pipe, a first switching hole is disposed on one side of the ventilation pipe located inside the bottom support pipe, a semicircular baffle plate is welded to one end of the bottom support pipe located inside the bottom support box, a blowing and suction pump is rotatably embedded inside the ventilation pipe, a semicircular pipe is mounted at one end of the blowing and suction pump located inside the ventilation pipe, a second switching hole is disposed on a side face of the semicircular pipe corresponding to the first switching hole, and an annular drying cage is placed inside the bottom support box;

an exhaust end of the blowing and suction pump is rotatably connected to one end of an air inspection pipe, and the other end of the air inspection pipe is connected to a gas inspection instrument; and

a top end of the bottom support box is movably clamped with a splice box, a splice pipe is welded to a bottom end of the splice box, a camera penetrates through and is mounted at a middle part of the splice box, an inspection light penetrates through and is mounted on the splice pipe close to the camera, an inspection pipe penetrates through and is mounted on one side of the bottom support box close to the storage box, and an inspection hole is disposed on the storage box corresponding to the inspection pipe.

According to the above technical solution, side vent holes are uniformly disposed at the bottom end of an outer side of the storage box, a windshield ring is slidably sleeved over the bottom end of the outer side of the storage box, a sliding clamping plate is movably sleeved over an outer side of the splice box, two ends of the sliding clamping plate are connected to the windshield ring by screws, one end of the sliding clamping plate is connected to one end of a connecting plate, and the other end of the connecting plate is connected to a transmission pipe; and a top end of the transmission pipe is welded to a docking head, a bottom end of the docking head is connected to a top end of an extendable end of an electric push rod, both the top end of the extendable end of the electric push rod and the top end of the transmission pipe are welded to the docking heads, an outer side of the docking head is abutted against an inner wall of the transmission pipe, and a positioning screw is mounted at a bottom end of the transmission pipe.

According to the above technical solution, a gear ring is sleeved over an outer side of the blowing and suction pump, one side of the gear ring is meshed to a gear, an output shaft end of a rotating motor is connected to the gear, the gear ring and the gear are located inside a gear box, the blowing and suction pump rotatably penetrates through the gear box, and the rotating motor is connected to an outer side of the gear box by screws.

According to the above technical solution, an outer side of the annular drying cage is abutted against an inner wall of the storage box, and the annular drying cage is filled with a mixture of silica gel desiccant and iron powder.

According to the above technical solution, a plurality of storage boxes are arranged, the bottom support ring forms a transition fit with a top end of each storage box, and an edge of a bottom end of the bottom support ring is rounded.

According to the above technical solution, input ends of the rotating motor, the gas inspection instrument, the camera, the inspection light and the electric push rod are electrically connected to corresponding output ends of an external power source, a cross bar is welded across a middle part of the inspection hole, and the camera and the inspection light are located on two sides of the cross bar.

According to the above technical solution, a switching auxiliary assembly is mounted inside each storage box and the bottom support box, and the switching auxiliary assembly includes a switching motor;

the switching motor is internally mounted at a middle part of the bottom end of the storage box, a top end of an output shaft of the switching motor is fixedly clamped with an outlet pipe, air delivery holes are symmetrically disposed at a bottom end of the outlet pipe, the outlet pipe penetrates through one end of a horizontal pipe, and an air delivery pump is connected to a bottom end of the other end of the horizontal pipe;

a transmission air pipe penetrates through and is rotatably mounted at a middle part of the storage box, hollow hexagonal blocks are welded to top ends of the transmission air pipe and the outlet pipe, a hexagonal hole is disposed at a bottom end of the transmission air pipe, a bottom disc is fixedly sleeved onto the transmission air pipe close to an internal bottom face of the storage box, fixing holes are uniformly disposed on the bottom face of the storage box, activity holes are disposed on the bottom disc corresponding to the fixing holes, partition frames are fixedly connected to an outer side of the transmission air pipe in a uniform manner, bladders are bonded to interiors of the partition frames, a check valve is mounted at one end of each bladder, one end of the check valve penetrates through a side face of the transmission air pipe, a bottom end of a transparent perforated pipe is connected to an outer side of the bottom disc, and a top end cover is placed at the uppermost storage box; and

an inner end cover is rotatably sleeved onto a top end of the transmission air pipe, clamping grooves are disposed at top ends of the partition frames corresponding to a bottom face of the inner end cover, and the bottom face of the inner end cover is uniformly bonded to cushioning air pads.

According to the above technical solution, input ends of the switching motor and the air delivery pump are electrically connected to corresponding output ends of the external power source, the switching motor and the air delivery pump are flush at bottom ends and are connected to the bottom support box by screws, and sealing gaskets are press-fitted onto top ends of the outlet pipe and the transmission air pipe.

According to the above technical solution, the fixing holes and the activity holes are identical in shape and size, and a contact surface between the bottom support box and the bottom disc is a smooth, flat surface.

According to the above technical solution, an application method for the device for predicting the storage period of fruits includes the steps of:

S1, inspection preparation: placing a bagged mixture of silica gel desiccant and iron powder into an annular drying cage, placing a bottom disc into a bottom support box, aligning fixing holes with activity holes at this time, and gently placing fruits to be stored sequentially on a top face of each bottom disc;

S2, placement and stacking: clamping an inspection pipe into an inspection hole, aligning a bottom support ring at a bottom end of each storage box with a top end of the bottom support box for stacking, docking a hexagonal hole at a bottom end of a transmission air pipe with a hollow hexagonal block at a top end of an outlet pipe, and mounting a gas inspection instrument at one end of an air inspection pipe;

S3, inspection and prediction: periodically opening each inspection light and camera to monitor fruit changes through optical observation, starting a switching motor to rotate each transmission air pipe, further rotating the fruits on the top face of each bottom disc for comprehensive observation and inspection, extracting air from each storage box using a blowing and suction pump, and directing the air to the gas inspection instrument for inspection; and

S4, environmental modification: starting a rotating motor to drive the blowing and suction pump to rotate 180° in a ventilation pipe, blowing air into the bottom support box by the blowing and suction pump, performing drying and deoxygenation treatment on the air, and exhausting the original air from each storage box, creating a low-oxygen, dry environment for fruit storage.

Compared to the related art, the present disclosure has the following beneficial effects.

1. The adjustment prediction assemblies are arranged. During storage, the inspection lights and the cameras are periodically opened to monitor fruit changes through optical observation. The rotating motor drives the blowing and suction pump to rotate inside the ventilation pipe, resulting in the semicircular baffle plate blocking an end portion of the semicircular pipe. At this time, the first switching hole is aligned with the second switching hole, causing the semicircular pipe to communicate internally with the bottom support box. Air within the storage boxes is extracted by the blowing and suction pump to the gas inspection instrument for inspection, which quantifies levels of ethylene and oxygen emitted by the fruits, as well as ethanol produced from fruit spoilage, thereby assessing current fruit conditions. Furthermore, the storage period is re-predicted based on visual changes and environmental fluctuations, and an initially predicted storage period is adjusted. With this simple operation, prediction accuracy and efficiency are improved.

When the storage period is reduced due to high humidity and oxygen levels in the storage boxes, the storage boxes are communicated with the bottom support box. The rotating motor drives the blowing and suction pump within the gear ring via the gear to rotate 180° inside the ventilation pipe, with no internal communication between the ventilation pipe and the bottom support box. Air is blown into the bottom support box using the blowing and suction pump, and the electric push rod is started, retracting the windshield rings to expose the side vent holes. The air is dried by silica gel desiccant and deoxygenated by iron powder in the annular drying cage. Furthermore, original air within the storage boxes is exhausted, creating a low-oxygen, dry environment and extending the predicted storage period of fruits.

2. The switching auxiliary assemblies are arranged. If frequent relocation of stored fruits is required, the air delivery pump can be started. The inflated bladders can compress the stored fruits, inducing mutual compression between adjacent fruits, thereby preventing impact-induced damage during relocation of stacked storage boxes caused by fruit-to-fruit collisions. Furthermore, the inflated bladders are flexible materials that preclude fruit damage from excessive pressure, thereby facilitating fruit storage.

The switching motor is started. The switching motor drives the transmission air pipes to rotate, further driving the fruits on the top face of each bottom disc to rotate, thereby causing the cameras to perform a comprehensive inspection of the fruits and enhancing inspection accuracy.

Upon completion of inspection and prediction operations, the switching motor is started. This switching motor drives the transmission air pipes to rotate, and the bottom discs rotate, causing the fixing holes and the activity holes to no longer align, which prevents mutual interference between the storage boxes, thereby facilitating fruit storage.

In summary, the adjustment prediction assembly can periodically monitor the current fruit conditions and change the internal environment of the storage boxes according to preservation requirements, thereby extending the predicted storage period. The switching auxiliary assembly can facilitate optimal fruit storage by minimizing fruit-to-fruit collisions and supplement the optical detection of the cameras, yielding more comprehensive and accurate inspection results to better support the adjustment prediction assembly. Through coordinated operation, the two assemblies can achieve superior detection accuracy and prediction precision.

### BRIEF DESCRIPTION OF THE DRAWINGS

Drawings, an integral part of the specification, are intended to provide a deeper understanding of the present disclosure and serve to explain the present disclosure along with embodiments, but do not limit the scope of the present disclosure.

In the accompanying drawings:

FIG. 1 is a schematic structural diagram of the present disclosure;

FIG. 2 is a schematic structural diagram of an adjustment prediction assembly of the present disclosure;

FIG. 3 is a schematic diagram showing a mounting structure of an inspection pipe of the present disclosure;

FIG. 4 is a schematic diagram showing a mounting structure of a bottom support pipe of the present disclosure;

FIG. 5 is a schematic diagram showing a mounting structure of a transmission pipe of the present disclosure;

FIG. 6 is a schematic structural diagram of a switching auxiliary assembly of the present disclosure;

FIG. 7 is a schematic diagram showing a mounting structure of an outlet pipe of the present disclosure;

FIG. 8 is a schematic diagram showing a mounting structure of a bladder of the present disclosure;

FIG. 9 is a schematic structural diagram of portion A in FIG. 8 of the present disclosure;

FIG. 10 is a schematic diagram showing a mounting structure of a cushioning air pad of the present disclosure; and

FIG. 11 is a flow chart of a method of the present disclosure.

Reference numerals and denotations thereof: 1—storage box;
2—adjustment prediction assembly; 201—bottom support ring; 202—bottom support box; 203—ventilation hole; 204—ventilation pipe; 205—bottom support pipe; 206—first switching hole; 207—semicircular baffle plate; 208—blowing and suction pump; 209—semicircular pipe; 210—second switching hole; 211—annular drying cage; 212—gear ring; 213—gear; 214—rotating motor; 215—gear box; 216—air inspection pipe; 217—gas inspection instrument; 218—splice box; 219—splice pipe; 220—camera; 221—inspection light; 222—inspection pipe; 223—inspection hole; 224—side vent hole; 225—windshield ring; 226—sliding clamping plate; 227—connecting plate; 228—transmission pipe; 229—docking head; and 230—electric push rod; and
3—switching auxiliary assembly; 301—switching motor; 302—outlet pipe; 303—air delivery hole; 304—horizontal pipe; 305—air delivery pump; 306—transmission air pipe; 307—hollow hexagonal block; 308—hexagonal hole; 309—bottom disc; 310—fixing hole; 311—activity hole; 312—partition frame; 313—bladder; 314—check valve; 315—transparent perforated pipe; 316—top end cover; 317—inner end cover; 318—clamping groove; 319—cushioning air pad; and 320—sealing gasket.

### DETAILED DESCRIPTION

The preferred embodiment of the present disclosure is described below in combination with the accompanying drawings. It is to be understood that the preferred embodiment is merely for illustration and explanation of the present disclosure, rather than limiting the present disclosure.

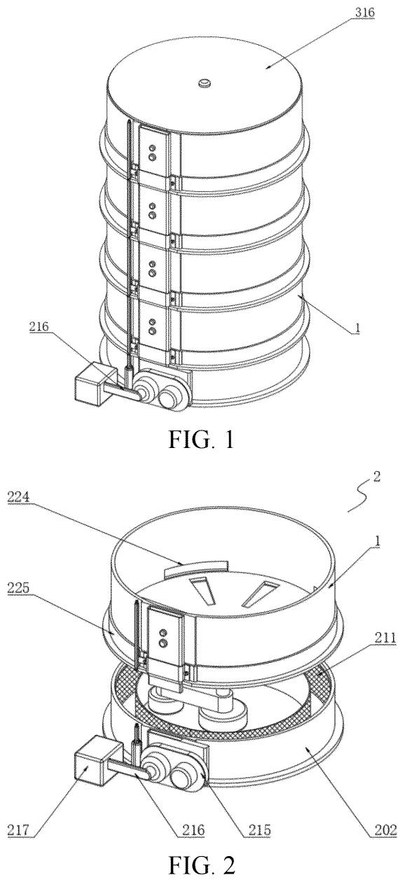

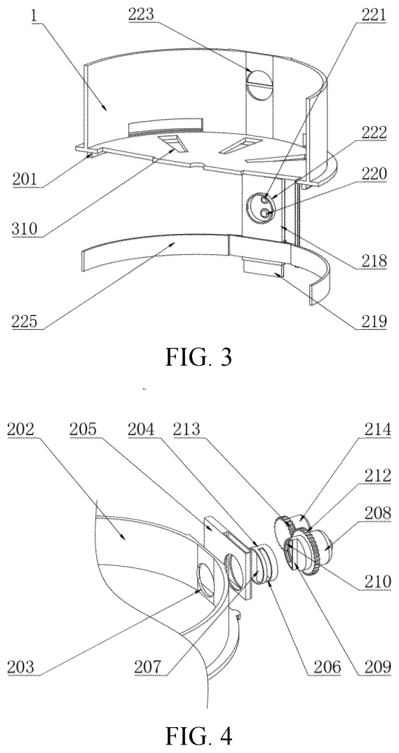

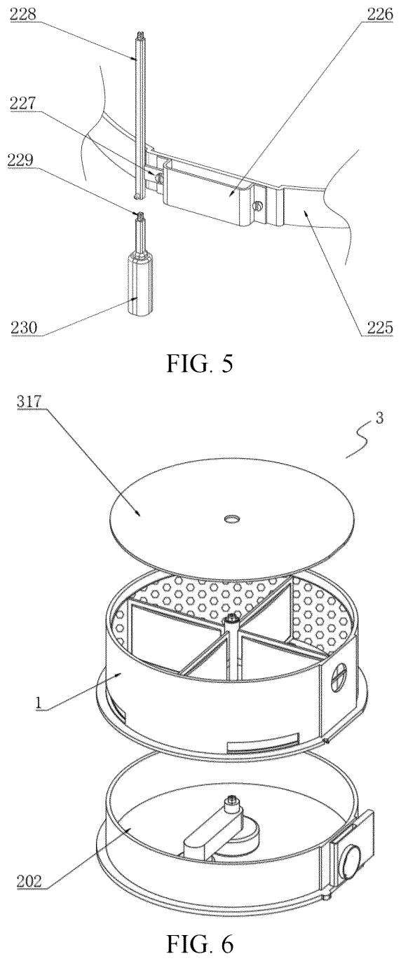

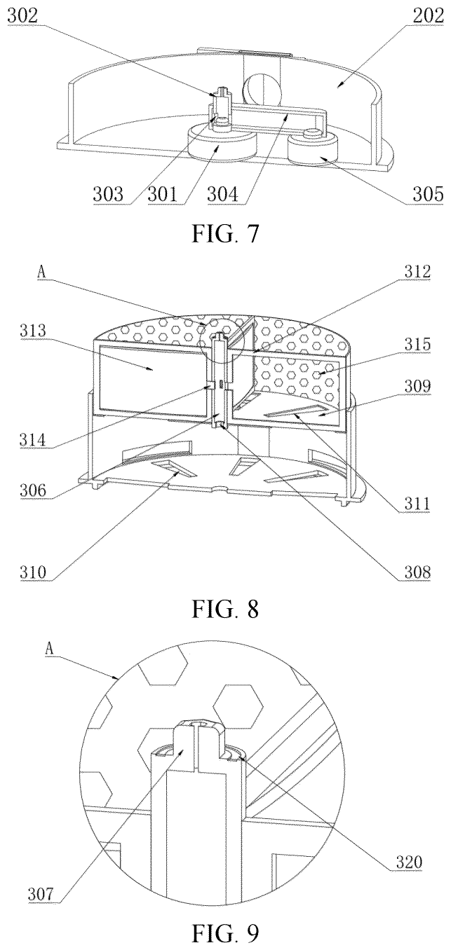

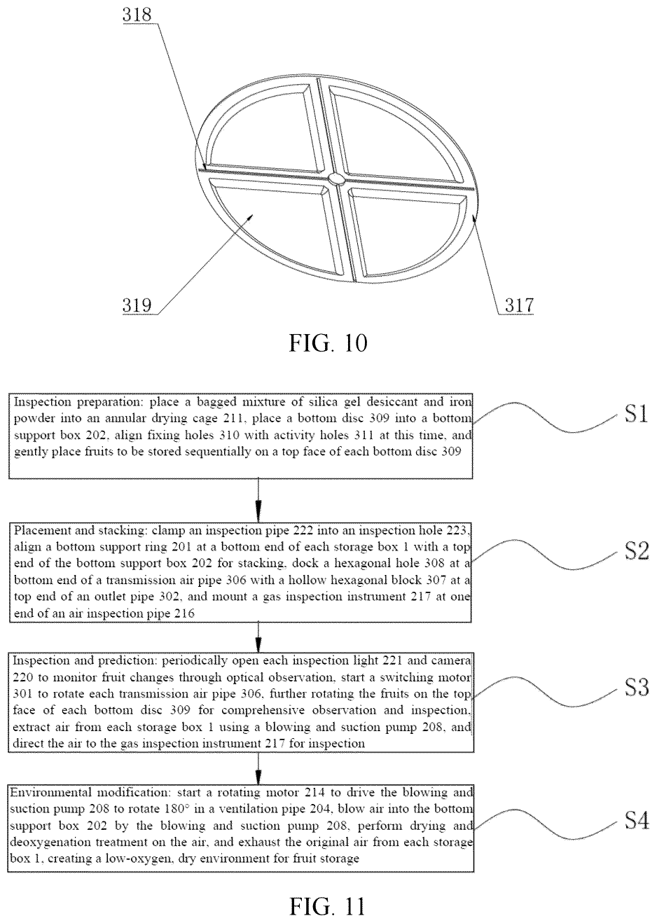

Embodiment: referring to FIGS. 1-10, the present disclosure provides a device for predicting the storage period of fruits, including storage boxes 1. An adjustment prediction assembly 2 is arranged on an outer side of each storage boxes 1, and the adjustment prediction assembly 2 includes a bottom support ring 201, a bottom support box 202, a ventilation hole 203, a ventilation pipe 204, a bottom support pipe 205, a first switching hole 206, a semicircular baffle plate 207, a blowing and suction pump 208, a semicircular pipe 209, a second switching hole 210, an annular drying cage 211, a gear ring 212, a gear 213, a rotating motor 214, a gear box 215, an air inspection pipe 216, a gas inspection instrument 217, a splice box 218, a splice pipe 219, a camera 220, an inspection light 221, an inspection pipe 222, an inspection hole 223, side vent holes 224, a windshield ring 225, a sliding clamping plate 226, a connecting plate 227, a transmission pipe 228, docking heads 229, and an electric push rod 230.

A bottom end of the storage box 1 is bonded to the bottom support ring 201, there are a plurality of storage boxes 1, the bottom support ring 201 forms a transition fit with a top end of each storage box 1, and an edge of a bottom end of the bottom support ring 201 is rounded to facilitate vertical stacking of the storage boxes 1 by docking the bottom support rings 201 with the storage boxes 1. The bottom support box 202 is movably sleeved over an outer side of the bottom support ring 201, the ventilation hole 203 is disposed on one side of the bottom support box 202, the ventilation pipe 204 is embedded and mounted inside the ventilation hole 203, the ventilation pipe 204 penetrates through a middle part of the bottom support pipe 205, the first switching hole 206 is disposed on one side of the ventilation pipe 204 located inside the bottom support pipe 205, the semicircular baffle plate 207 is welded to one end of the bottom support pipe 205 located inside the bottom support box 202, the blowing and suction pump 208 is rotatably embedded inside the ventilation pipe 204, the semicircular pipe 209 is mounted at one end of the blowing and suction pump 208 located inside the ventilation pipe 204, the second switching hole 210 is disposed on a side face of the semicircular pipe 209 corresponding to the first switching hole 206, and the annular drying cage 211 is placed inside the bottom support box 202. An outer side of the annular drying cage 211 is abutted against an inner wall of the storage box 1, and the annular drying cage 211 is filled with a mixture of silica gel desiccant and iron powder, facilitating dehumidification and deoxygenation of introduced air streams.

The gear ring 212 is sleeved over an outer side of the blowing and suction pump 208, one side of the gear ring 212 is meshed to the gear 213, an output shaft end of the rotating motor 214 is connected to the gear 213, the gear ring 212 and the gear 213 are located inside the gear box 215, the blowing and suction pump 208 rotatably penetrates through the gear box 215, and the rotating motor 214 is connected to an outer side of the gear box 215 by screws, facilitating the mounting of the rotating motor 214 while protecting the gear ring 212 and gear 213. An exhaust end of the blowing and suction pump 208 is rotatably connected to one end of the air inspection pipe 216, and the other end of the air inspection pipe 216 is connected to the gas inspection instrument 217.

A top end of the bottom support box 202 is movably clamped with the splice box 218, the splice pipe 219 is welded to a bottom end of the splice box 218, the camera 220 penetrates through and is mounted at a middle part of the splice box 218, the inspection light 221 penetrates through and is mounted on the splice pipe 219 close to the camera 220, the inspection pipe 222 penetrates through and is mounted on one side of the bottom support box 202 close to the storage box 1, and the inspection hole 223 is disposed on the storage box 1 corresponding to the inspection pipe 222.

The side vent holes 224 are uniformly disposed at the bottom end of an outer side of the storage box 1, the windshield ring 225 is slidably sleeved over the bottom end of the outer side of the storage box 1, the sliding clamping plate 226 is movably sleeved over an outer side of the splice box 218, two ends of the sliding clamping plate 226 are connected to the windshield ring 225 by screws, one end of the sliding clamping plate 226 is connected to one end of the connecting plate 227, and the other end of the connecting plate 227 is connected to the transmission pipe 228; and a top end of the transmission pipe 228 is welded to the docking head 229, and a bottom end of the docking head 229 is connected to a top end of an extendable end of an electric push rod 230. Input ends of the rotating motor 214, the gas inspection instrument 217, the camera 220, the inspection light 221 and the electric push rod 230 are electrically connected to corresponding output ends of an external power source, a cross bar is welded across a middle part of the inspection hole 223, and the camera 220 and the inspection light 221 are located on two sides of the cross bar, ensuring the normal operation of the rotating motor 214, the gas inspection instrument 217, the camera 220, the inspection light 221, and the electric push rod 230. Both the top end of the extendable end of the electric push rod 230 and the top end of the transmission pipe 228 are welded to the docking heads 229, an outer side of the docking head 229 is abutted against an inner wall of the transmission pipe 228, and a positioning screw is mounted at a bottom end of the transmission pipe 228.

A switching auxiliary assembly 3 is mounted inside each storage box 1 and the bottom support box 202, and the switching auxiliary assemblies 3 include a switching motor 301, an outlet pipe 302, air delivery holes 303, a horizontal pipe 304, an air delivery pump 305, transmission air pipes 306, hollow hexagonal blocks 307, hexagonal holes 308, bottom discs 309, fixing holes 310, activity holes 311, partition frames 312, bladders 313, check valves 314, transparent perforated pipes 315, a top end cover 316, inner end covers 317, clamping grooves 318, cushioning air pads 319, and sealing gaskets 320.

the switching motor 301 is internally mounted at a middle part of the bottom end of the storage box 1, a top end of an output shaft of the switching motor 301 is fixedly clamped with the outlet pipe 302, the air delivery holes 303 are symmetrically disposed at a bottom end of the outlet pipe 302, the outlet pipe 302 penetrates through one end of the horizontal pipe 304, and the air delivery pump 305 is connected to a bottom end of the other end of the horizontal pipe 304. Input ends of the switching motor 301 and the air delivery pump 305 are electrically connected to corresponding output ends of the external power source, the switching motor 301 and the air delivery pump 305 are flush at bottom ends and are connected to the bottom support box 202 by screws, and sealing gaskets 320 are press-fitted onto top ends of the outlet pipe 302 and the transmission air pipe 306, ensuring the sealing performance at the connection of the transmission air pipe 306 and guaranteeing the normal operation of the switching motor 301 and the air delivery pump 305.

The transmission air pipe 306 penetrates through and is rotatably mounted at a middle part of the storage box 1, the hollow hexagonal blocks 307 are welded to top ends of the transmission air pipe 306 and the outlet pipe 302, the hexagonal hole 308 is disposed at a bottom end of the transmission air pipe 306, the bottom disc 309 is fixedly sleeved onto the transmission air pipe 306 close to an internal bottom face of the storage box 1, the fixing holes 310 are uniformly disposed on the bottom face of the storage box 1, and the activity holes 311 are disposed on the bottom disc 309 corresponding to the fixing holes 310. The fixing holes 310 and the activity holes 311 are identical in shape and size, and a contact surface between the bottom support box 202 and the bottom disc 309 is a smooth, flat surface, facilitating the rotation of the bottom disc 309 within the bottom support box 202, and the alignment of the fixing holes 310 with the activity holes 311. The partition frames 312 are fixedly connected to an outer side of the transmission air pipe 306 in a uniform manner, the bladders 313 are bonded to interiors of the partition frames 312, the check valve 314 is mounted at one end of each bladder 313, one end of the check valve 314 penetrates through a side face of the transmission air pipe 306, a bottom end of the transparent perforated pipe 315 is connected to an outer side of the bottom disc 309, and the top end cover 316 is placed at the uppermost storage box 1. The inner end cover 317 is rotatably sleeved onto a top end of the transmission air pipe 306, the clamping grooves 318 are disposed at top ends of the partition frames 312 corresponding to a bottom face of the inner end cover 317, and the bottom face of the inner end cover 317 is uniformly bonded to the cushioning air pads 319.

Referring to FIG. 11, an application method for the device for predicting the storage period of fruits includes the steps that:

In S1, inspection preparation: a bagged mixture of silica gel desiccant and iron powder is placed into an annular drying cage 211, and a bottom disc 309 is placed into a bottom support box 202. At this time, fixing holes 310 are aligned with activity holes 311, and fruits to be stored are gently placed sequentially on a top face of each bottom disc 309.

In S2, placement and stacking: an inspection pipe 222 is clamped into an inspection hole 223. A bottom support ring 201 at a bottom end of each storage box 1 is aligned with a top end of the bottom support box 202 for stacking, and a hexagonal hole 308 at a bottom end of a transmission air pipe 306 is docked with a hollow hexagonal block 307 at a top end of an outlet pipe 302. Moreover, a gas inspection instrument 217 is mounted at one end of an air inspection pipe 216.

In S3, inspection and prediction: each inspection light 221 and camera 220 are periodically opened to monitor fruit changes through optical observation. A switching motor 301 is started to rotate each transmission air pipe 306, further rotating the fruits on the top face of each bottom disc 309 for comprehensive observation and inspection. Air from each storage box 1 is extracted using a blowing and suction pump 208, and directed to the gas inspection instrument 217 for inspection.

In S4, environmental modification: a rotating motor 214 is started to drive the blowing and suction pump 208 to rotate 180° in a ventilation pipe 204. Air is blown into the bottom support box 202 by the blowing and suction pump 208. Drying and deoxygenation treatment is performed on the air, and the original air is exhausted from each storage box 1, creating a low-oxygen, dry environment for fruit storage.

An operating principle and operational process of the present disclosure are as follows. The bottom support box 202 is placed at a designated position, and the bagged mixture of silica gel desiccant and iron powder is loaded into the annular drying cage 211 to ensure sufficient gaps within the annular drying cage 211, thereby facilitating air circulation. Moreover, the bottom disc 309 is placed into the bottom support box 202. At this time, the fixing holes 310 are aligned with the activity holes 311, and the fruits to be stored are gently placed sequentially on the top face of each bottom disc 309, with the skins inspected for integrity and absence of damage during placement. The inner end covers 317 are sleeved onto the top ends of the transmission air pipes 306, and the clamping grooves 318 are clamped with the top ends of the partition frames 312.

The inspection pipe 222 is clamped into the inspection hole 223. Subsequently, the bottom support ring 201 at the bottom end of each storage box 1 is aligned with the top end of the bottom support box 202 for stacking. A splice pipe 219 is embedded into the top end of the bottom support pipe 205, and the hexagonal hole 308 at the bottom end of the transmission air pipe 306 is docked with the hollow hexagonal block 307 at the top end of the outlet pipe 302. Subsequently, the plurality of storage boxes 1 are sequentially stacked, a top end of the topmost splice box 218 is sealed, and the top end cover 316 is covered onto the topmost storage box 1. Moreover, the gas inspection instrument 217 is mounted at one end of the air inspection pipe 216, and all electric devices are connected to a control platform for subsequent operational convenience.

During storage, if frequent relocation of stored fruits is required, the air delivery pump 305 can be started. Air sequentially passes through the horizontal pipe 304, the air delivery holes 303, the outlet pipe 302, the transmission air pipes 306, and the check valves 314 before entering the bladders 313. The inflated bladders 313 can compress the stored fruits, inducing mutual compression between adjacent fruits, thereby preventing impact-induced damage during relocation of stacked storage boxes 1 caused by fruit-to-fruit collisions. Furthermore, the inflated bladders 313 are flexible materials that preclude fruit damage from excessive pressure, thereby facilitating fruit storage.

During storage, the inspection lights 221 and the cameras 220 are periodically opened to monitor fruit changes through optical observation. The rotating motor 214 is started, and the rotating motor 214 drives the blowing and suction pump 208 within the gear ring 212 via the gear 213 to rotate inside the ventilation pipe 204, causing one end of the semicircular pipe 209 to align with one side of the semicircular baffle plate 207, resulting in the semicircular baffle plate 207 blocking an end portion of the semicircular pipe 209. At this time, the first switching hole 206 is aligned with the second switching hole 210, causing the semicircular pipe 209 to communicate internally with the bottom support box 202. Simultaneously, since each storage box 1 is communicated with the spliced splice box 218 via the air inspection pipe 216, air within the storage boxes 1 is extracted by the blowing and suction pump 208 to the gas inspection instrument 217 for inspection, which quantifies levels of ethylene and oxygen emitted by the fruits, as well as ethanol produced from fruit spoilage, thereby assessing current fruit conditions. Furthermore, the storage period is re-predicted based on visual changes and environmental fluctuations, and an initially predicted storage period is adjusted. With this simple operation, prediction accuracy and efficiency are improved.

During the above inspection process, the switching motor 301 can be started. The switching motor 301 drives the transmission air pipes 306 to rotate, further driving the fruits on the top face of each bottom disc 309 to rotate, thereby causing the cameras 220 to perform a comprehensive inspection of the fruits and enhancing inspection accuracy.

After inspection and prediction, when the storage period is reduced due to high humidity and oxygen levels in the storage boxes 1, the storage boxes 1 are communicated with the bottom support box 202 to extend the storage period. The rotating motor 214 is started, and the rotating motor 214 drives the blowing and suction pump 208 within the gear ring 212 via the gear 213 to rotate 180° inside the ventilation pipe 204, causing the semicircular pipe 209 and the semicircular baffle plate 207 to no longer align, resulting in the semicircular baffle plate 207 no longer blocking the end portion of the semicircular pipe 209. At this time, the first switching hole 206 and the second switching hole 210 no longer align, with no internal communication with the bottom support box 202. The air is blown into the bottom support box 202 by the blowing and suction pump 208. Moreover, the electric push rod 230 is started to push the transmission pipes 228 upward, causing the windshield rings 225 to no longer block the side vent holes 224, thereby preventing excessive air pressure within the storage boxes 1. Dried by silica gel desiccant and deoxygenated by iron powder in the annular drying cage 211, the air becomes dry, low-oxygen content gas. Furthermore, the treated gas sequentially enters the storage boxes 1 from the bottom end, and the original air within the storage boxes 1 is exhausted, creating a low-oxygen, dry environment and extending the predicted storage period of fruits.

Upon completion of inspection and prediction operations, the switching motor 301 is started. This switching motor 301 drives the transmission air pipes 306 to rotate, and the bottom discs 309 rotates, causing the fixing holes 310 and the activity holes 311 to no longer align, which prevents mutual interference between the storage boxes 1, thereby facilitating fruit storage.

The adjustment prediction assembly 2 can periodically monitor the current fruit conditions and change the internal environment of the storage boxes 1 according to preservation requirements, thereby extending the predicted storage period. The switching auxiliary assembly 3 can facilitate optimal fruit storage by minimizing fruit-to-fruit collisions, and supplement the optical detection of the cameras 220, yielding more comprehensive and accurate inspection results to better support the adjustment prediction assembly 2. Through coordinated operation, the two assemblies can achieve superior detection accuracy and prediction precision.

Finally, it is to be noted that the foregoing is only the preferred embodiment of the present disclosure, rather than limiting the present disclosure. Although the present disclosure has been described by the above embodiments in detail, a person of ordinary skilled in the art may still modify the technical solutions described in the above embodiments, or perform equivalent replacement of some technical features thereof. Any modification, equivalent substitution, improvement, and the like made within the spirit and principles of the present disclosure shall be included within the scope of protection of the present disclosure.

1. A device for predicting the storage period of fruits, comprising storage boxes (1), wherein an adjustment prediction assembly (2) is arranged on an outer side of each storage box (1), and the adjustment prediction assembly (2) comprises a bottom support ring (201);
a bottom end of the storage box (1) is bonded to the bottom support ring (201), a bottom support box (202) is movably sleeved over an outer side of the bottom support ring (201), a ventilation hole (203) is disposed on one side of the bottom support box (202), a ventilation pipe (204) is embedded and mounted inside the ventilation hole (203), the ventilation pipe (204) penetrates through a middle part of a bottom support pipe (205), and a first switching hole (206) is disposed on one side of the ventilation pipe (204) located inside the bottom support pipe (205); and a semicircular baffle plate (207) is welded to one end of the bottom support pipe (205) located inside the bottom support box (202), a blowing and suction pump (208) is rotatably embedded inside the ventilation pipe (204), a semicircular pipe (209) is mounted at one end of the blowing and suction pump (208) located inside the ventilation pipe (204), a second switching hole (210) is disposed on a side face of the semicircular pipe (209) corresponding to the first switching hole (206), and an annular drying cage (211) is placed inside the bottom support box (202);
an exhaust end of the blowing and suction pump (208) is rotatably connected to one end of an air inspection pipe (216), and the other end of the air inspection pipe (216) is connected to a gas inspection instrument (217); and
a top end of the bottom support box (202) is movably clamped with a splice box (218), a splice pipe (219) is welded to a bottom end of the splice box (218), a camera (220) penetrates through and is mounted at a middle part of the splice box (218), an inspection light (221) penetrates through and is mounted on the splice pipe (219) close to the camera (220), an inspection pipe (222) penetrates through and is mounted on one side of the bottom support box (202) close to the storage box (1), and an inspection hole (223) is disposed on the storage box (1) corresponding to the inspection pipe (222).

2. The device for predicting the storage period of fruits according to claim 1, wherein side vent holes (224) are uniformly disposed at the bottom end of an outer side of the storage box (1), a windshield ring (225) is slidably sleeved over the bottom end of the outer side of the storage box (1) a sliding clamping plate (226) is movably sleeved over an outer side of the splice box (218), two ends of the sliding clamping plate (226) are connected to the windshield ring (225) by screws, one end of the sliding clamping plate (226) is connected to one end of a connecting plate (227), and the other end of the connecting plate (227) is connected to a transmission pipe (228); and a top end of the transmission pipe (228) is welded to a docking head (229), a bottom end of the docking head (229) is connected to a top end of an extendable end of an electric push rod (230), both the top end of the extendable end of the electric push rod (230) and the top end of the transmission pipe (228) are welded to the docking heads (229), an outer side of the docking head (229) is abutted against an inner wall of the transmission pipe (228), and a positioning screw is mounted at a bottom end of the transmission pipe (228).

3. The device for predicting the storage period of fruits according to claim 2, wherein a gear ring (212) is sleeved over an outer side of the blowing and suction pump (208), one side of the gear ring (212) is meshed to a gear (213), an output shaft end of a rotating motor (214) is connected to the gear (213), the gear ring (212) and the gear (213) are located inside a gear box (215), the blowing and suction pump (208) rotatably penetrates through the gear box (215), and the rotating motor (214) is connected to an outer side of the gear box (215) by screws.

4. The device for predicting the storage period of fruits according to claim 1, wherein an outer side of the annular drying cage (211) is abutted against an inner wall of the storage box (1), and the annular drying cage (211) is filled with a mixture of silica gel desiccant and iron powder.

5. The device for predicting the storage period of fruits according to claim 1, wherein a plurality of storage boxes (1) are arranged, the bottom support ring (201) forms a transition fit with a top end of each storage box (1), and an edge of a bottom end of the bottom support ring (201) is rounded.

6. The device for predicting the storage period of fruits according to claim 3, wherein input ends of the rotating motor (214), the gas inspection instrument (217), the camera (220), the inspection light (221) and the electric push rod (230) are electrically connected to corresponding output ends of an external power source, a cross bar is welded across a middle part of the inspection hole (223), and the camera (220) and the inspection light (221) are located on two sides of the cross bar.

7. The device for predicting the storage period of fruits according to claim 6, wherein a switching auxiliary assembly (3) is mounted inside each storage box (1) and the bottom support box (202), and the switching auxiliary assembly (3) comprises a switching motor (301); the switching motor (301) is internally mounted at a middle part of the bottom end of the storage box (1), a top end of an output shaft of the switching motor (301) is fixedly clamped with an outlet pipe (302), air delivery holes (303) are symmetrically disposed at a bottom end of the outlet pipe (302), the outlet pipe (302) penetrates through one end of a horizontal pipe (304), and an air delivery pump (305) is connected to a bottom end of the other end of the horizontal pipe (304); and a transmission air pipe (306) penetrates through and is rotatably mounted at a middle part of the storage box (1), hollow hexagonal blocks (307) are welded to top ends of the transmission air pipe (306) and the outlet pipe (302), a hexagonal hole (308) is disposed at a bottom end of the transmission air pipe (306), a bottom disc (309) is fixedly sleeved onto the transmission air pipe (306) close to an internal bottom face of the storage box (1), fixing holes (310) are uniformly disposed on the bottom face of the storage box (1), activity holes (311) are disposed on the bottom disc (309) corresponding to the fixing holes (310), partition frames (312) are fixedly connected to an outer side of the transmission air pipe (306) in a uniform manner, bladders (313) are bonded to interiors of the partition frames (312), a check valve (314) is mounted at one end of each bladder (313), one end of the check valve (314) penetrates through a side face of the transmission air pipe (306), a bottom end of a transparent perforated pipe (315) is connected to an outer side of the bottom disc (309), and a top end cover (316) is placed at the uppermost storage box (1); and
an inner end cover (317) is rotatably sleeved onto a top end of the transmission air pipe (306), clamping grooves (318) are disposed at top ends of the partition frames (312) corresponding to a bottom face of the inner end cover (317), and the bottom face of the inner end cover (317) is uniformly bonded to cushioning air pads (319).

8. The device for predicting the storage period of fruits according to claim 7, wherein input ends of the switching motor (301) and the air delivery pump (305) are electrically connected to corresponding output ends of the external power source, the switching motor (301) and the air delivery pump (305) are flush at bottom ends and are connected to the bottom support box (202) by screws, and sealing gaskets (320) are press-fitted onto top ends of the outlet pipe (302) and the transmission air pipe (306).

9. The device for predicting the storage period of fruits according to claim 7, wherein the fixing holes (310) and the activity holes (311) are identical in shape and size, and a contact surface between the bottom support box (202) and the bottom disc (309) is a smooth, flat surface.

10. An application method for the device for predicting the storage period of fruits according to claim 7, comprising the steps of:
S1, inspection preparation: placing a bagged mixture of silica gel desiccant and iron powder into an annular drying cage (211), placing a bottom disc (309) into a bottom support box (202), aligning fixing holes (310) with activity holes (311) at this time, and gently placing fruits to be stored sequentially on a top face of each bottom disc (309);
S2, placement and stacking: clamping an inspection pipe (222) into an inspection hole (223), aligning a bottom support ring (201) at a bottom end of each storage box (1) with a top end of the bottom support box (202) for stacking, docking a hexagonal hole (308) at a bottom end of a transmission air pipe (306) with a hollow hexagonal block (307) at a top end of an outlet pipe (302), and mounting a gas inspection instrument (217) at one end of an air inspection pipe (216);
S3, inspection and prediction: periodically opening each inspection light (221) and camera (220) to monitor fruit changes through optical observation, starting a switching motor (301) to rotate each transmission air pipe (306), further rotating the fruits on the top face of each bottom disc (309) for comprehensive observation and inspection, extracting air from each storage box (1) using a blowing and suction pump (208), and directing the air to the gas inspection instrument (217) for inspection; and
S4, environmental modification: starting a rotating motor (214) to drive the blowing and suction pump (208) to rotate 180°in a ventilation pipe (204), blowing air into the bottom support box (202) by the blowing and suction pump (208), performing drying and deoxygenation treatment on the air, and exhausting the original air from each storage box (1), creating a low-oxygen, dry environment for fruit storage.
